SEQUENCE LISTING (8 sequences)

SEQ ID NO: 1 - DNA, 20 bp, Artificial Sequence
  primer
gcaacagttactgcgacgtg

SEQ ID NO: 2 - DNA, 20 bp, Artificial Sequence
  primer
ggacacagtggcttttgaca

SEQ ID NO: 3 - DNA, 20 bp, Artificial Sequence
  primer
gatggtccagctggacaagc

SEQ ID NO: 4 - DNA, 20 bp, Artificial Sequence
  primer
gtgcccattaacaggtgttc

SEQ ID NO: 5 - DNA, 25 bp, Artificial Sequence
  primer
cgrgaagctggagaaggagaagctg

SEQ ID NO: 6 - DNA, 24 bp, Artificial Sequence
  primer
aagggccgcagcttacacatgttc

SEQ ID NO: 7 - DNA, 25 bp, Artificial Sequence
  primer
tggcaccacaccttctacaatgagc

SEQ ID NO: 8 - DNA, 25 bp, Artificial Sequence
  primer
gcacagcttctccttaatgtcacgc

(19) United States
(12) Patent Application Publication
(10) Pub. No.: US 2009/0029461 A1
(43) Pub. Date: Jan. 29, 2009

(54) Immortalised Feeder Cells

(75) Inventors: Andre Boon Hwa Choo, Singapore (SG); Steve Kah Weng Oh, Singapore (SG)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: AGENCY FOR SCIENCE TECHNOLOGY AND RESEARCH, SINGAPORE (SG)

(21) Appl. No.: 11/885,106

(22) PCT Filed: Feb. 23, 2006
(86) PCT No.: PCT/AU2006/000220

§ 371 Date: Sep. 2, 2008

(30) Foreign Application Priority Data
Feb. 23, 2005 (AU) 2005900842

(51) Int. Cl. C12N 5/06; C12N 5/08

(52) U.S. Cl. 435/357; 435/325; 435/373; 435/366

(57) ABSTRACT

The invention relates to an immortalized feeder cell line. The immortalized feeder cell line may be derived from an embryonic fibroblast, which may be a mouse embryonic fibroblast. A culture of an immortalized feeder cell line according to the invention in a suitable culture medium is also provided, as is a composition including an immortalized feeder cell line according to the invention in a suitable carrier or diluent and conditioned medium produced from growth of an immortalized feeder cell line according to the invention. The invention further provides a method of culturing a stem cell including use of a cell line or conditioned medium according to the invention and cells so produced.

### TECHNICAL FIELD

The present invention relates to immortalized feeder cell lines, their culture medium and the use of these cells and their medium in culturing stem cells, in particular, human embryonic stem cells.

### BACKGROUND

Since the successful isolation of human embryonic stem cells (hESC) from the inner cell mass of pre-implantation embryos, it is now possible to culture undifferentiated hESC indefinitely in vitro and thereby potentially provide a starting source of material that can be differentiated into cells from the three embryonic germ layers to be used in regenerative therapy [1] Conventionally, hESC are maintained directly on feeder layers as co-cultures and these feeders have been derived either from mouse or human sources [2-7]. More recently, hESC have been successful cultured under feeder-free conditions. Here, the cells are grown on extracellular matrices, such as matrigel and supplemented with conditioned medium (CM) from feeder layers [8,9]. Regardless of the source, feeders are derived from primary tissues and hence have a limited lifespan in culture. For example, primary feeders can only be cultured for approximately 7-9 passages before the cells senesce. Hence, fresh batches of feeders have to be prepared on a routine basis which may result in batch to batch variation. Furthermore, there are also concerns that pathogens may be transmitted from the feeders to the hESC.

One possible solution to obtaining a sustainable, validated and consistent source of feeders for the culture of hESC, especially for scale-up, is the immortalization of primary feeders. Several methods have been previously used for immortalization and these include the transduction of normal cells with genes from DNA tumor viruses such as simian virus 40 (SV4O), Epstein-Barr virus (EBV) and human papillomavirus (HPV) [10-14]. More recently, groups have also reported that the over-expression of the human telomerase reverse transcriptase, hTERT was also able to extend the lifespan of somatic or differentiated cells [15,16].

### DESCRIPTION OF THE INVENTION

In a first aspect the present invention provides an immortalized feeder cell line. In particular the present invention provides an immortalized feeder cell line derived from an embryonic fibroblast. More particularly the immortalized feeder cell line is derived from a mouse embryonic fibroblast. More particularly the immortalized feeder cell line is derived from a mouse embryonic fibroblast by one or more of degradation of the p53 tumor suppressor protein, up-regulation of c-myc expression, activation of telomerase and degradation of pRb. More particularly the immortalized feeder cell line is derived from an embryonic fibroblast by introduction of the E6 and E7 genes from HPV16. Preferably, the E6 and E7 genes are introduced by transduction. Preferably the embryonic fibroblast is infected with retrovirus vectors encoding the E6 and E7 genes from HPV16. Preferably, the bulk of the cells continue to proliferate beyond their normal lifespan and are resistant to antibiotic selection with G418 following infection with the retroviruses. Preferably the cell lines do not become tumorigenic after immortalization. Preferably, in vivo, intramuscular injection of the cells into SCID does not result in any palpable tumors even 16 weeks after injection.

Cell lines of the invention proliferate beyond 7, 8 or 9 passages. Preferred cell lines of the invention proliferate in vitro beyond 70 passages and do not gain any tumorigenic phenotype. Cell lines of the invention support hESC which: continue to maintain characteristic undifferentiated morphology for >40 passages both in co-culture and in feeder-free cultures supplemented with CM; continue to express the pluripotent markers, Oct-4, SSEA-4, Tra-1-60, Tra-1-81, alkaline phosphatase and maintain a normal karyotype; and when injected into SCID mice form teratomas with tissues representative of the 3 embryonic germ layers.

The cell lines of the invention support undifferentiated hESC growth. hESC lines readily adapt to these feeders and maintain the typical morphology of undifferentiated hESC cultures both in feeder and feeder-free cultures. The hESC also continued to express pluripotent markers, including Oct-4, SSEA-4, Tra-1-60, Tra-1-81 and alkaline phosphatase. Preferably after 25 passages, the cells retain a stable karyotype and are able to differentiate to form teratomas in SCID mice. Preferably, RT-PCR analysis of mRNA from hESC feeder-free cultures confirms that the cells remained positive for Oct-4 but negative for E6 and E7 antigens.

The invention provides primary MEF immortalized with the over-expression of the E6 and E7 antigens. In a preferred embodiment the cell line is ΔE-MEF as herein described.

The invention also provides a culture of an immortalized feeder cell line of the first aspect in a suitable culture medium and a composition including an immortalized feeder cell line of the first aspect in a suitable carrier or diluent.

In a second aspect the invention provides conditioned medium produced from growth of an immortalized feeder cell line of the first aspect.

The conditioned medium may be used in conjunction with extracellular matrices, such as matrigel.

In a third aspect the invention provides a method of culturing a stem cell including use of a cell line of the first aspect as feeder cells. Preferably the stem cell is a human stem cell. More preferably the stem cell is a human embryonic stem cell.

In a fourth aspect the invention provides a method of culturing a stem cell including use of a conditioned medium of the second aspect. Preferably the stem cell is a human stem cell. More preferably the stem cell is a human embryonic stem cell.

The methods of the third and fourth aspects include scaled-up quantities of undifferentiated hESC in culture vessels such as cell factories. The scaled up quantities may be >10⁸ cells.

In a fifth aspect the present invention provides a stem cell cultured by a method of the third or fourth aspect of the invention. Preferably the stem cell is a human stem cell. More preferably the stem cell is a human embryonic stem cell.

Human embryonic stem cells (hESC) are pluripotent cells that have the potential to proliferate indefinitely in culture, and still retain their capacity for differentiation into a wide variety of cells. However, hESC cultures require either feeders in direct contact or conditioned medium (CM) from feeders. The most common source of feeders is primary mouse embryonic fibroblast (MEF).

In this study, we investigated whether we could extend the proliferative capacity of primary MEF after infection with retrovirus vectors encoding the E6 and E7 genes from HPV16 beyond its normal lifespan in vitro. The over-expression of the E6 protein causes the degradation of the p53 tumor suppressor protein, as well as the up-regulation of c-myc expression and the activation of telomerase [17]. On the other hand, expressed E7 protein binds to the retinoblastoma protein, pRb resulting in the degradation of pRb. [18]. It has previously been shown that the expression of both E6 and E7 can efficiently immortalize normal human fibroblast, mammary epithelial cells and foreskin keratinocytes [11, 19, 20]. Here, we successfully immortalized primary MEF with the over-expression of the E6 and E7 antigens. The immortalized MEF, ΔE-MEF, continued to proliferate in vitro beyond 70 passages and this did not result in the cells gaining any tumorigenic phenotype. In addition, 3 hESC lines previously grown on primary MEF either on feeders or feeder-free conditions readily adapted to ΔE-MEF.

Morphologically, the hESC remained undifferentiated and continued to express both intracellular and extracellular markers characteristic of pluripotency. The hESC cultures also retained normal karyotype and formed teratomas in SCID mouse models.

In this study, we immortalized a primary MEF line by infection with retrovirus vectors encoding the E6 and E7 genes from HPV16. The immortal line, ΔE-MEF, was able to proliferate beyond 7-9 passages, while primary cells senesce, and have an extended lifespan beyond 70 passages. When tested for its ability to support hESC growth, it was found that hESC continue to maintain the characteristic undifferentiated morphology for >40 passages both in co-culture with ΔE-MEF and in feeder-free cultures supplemented with CM from ΔE-MEF. The cultures also continue to express the pluripotent markers, Oct-4, SSEA-4, Tra-1-60, Tra-1-81, alkaline phosphatase and maintain a normal karyotype. In addition, these hESC when injected into SCID mice formed teratomas with tissues representative of the 3 embryonic germ layers. Lastly, it was also demonstrated that it is possible to scale-up significant quantities of undifferentiated hESC (>10⁸ cells) using ΔE-MEF in culture vessels such as cell factories. The results from this study suggest that immortalized feeders can provide a consistent and reproducible source of feeders for hESC expansion and research and thus have an advantage over primary feeders.

Morphologically, the hESC remained undifferentiated and continued to express both intracellular and extracellular markers characteristic of pluripotency. The hESC cultures also retained normal karyotype and formed teratomas in SCID mouse models.

### BRIEF DESCRIPTION OF THE ACCOMPANYING FIGURES

FIG. 1. Immortalization of primary MEF. (a): Growth curve of MEF infected with retrovirus expressing E6/E7 (ΔE-MEF) at passage 7 (□) and passage 45 (Δ) or MEF infected with control retrovirus (AC-MEF) at passage 7 (▪). Culture samples were harvested daily and cell numbers were determined using Trypan blue exclusion. (b): RT-PCR analysis for the expression of HPV-E6 and HPV-E7 genes in E6/E7 infected cells (ΔE-MEF, lanes 2 and 4) and control virus infected cells (ΔC-MEF, lanes 1 and 3). Amplified products were resolved on a 1% agarose gel. MW, 100-bp DNA ladder (Promega).

FIG. 2. Morphology of the hESC line, HES-3 cells cultured directly on ΔE-MEF (left panel) or on Matrigel supplemented with conditioned media from ΔE-MEF (right panel). Representative images of the colonies taken using a stereomicroscope at 40× magnification (a and b, scale bar=400 μm) and phase-contrast microscope at 40× (c and d, scale bar=300 μm) and 100× (e, scale bar=100 μm) magnifications.

FIG. 3. Flow cytometry analysis of intracellular Oct-4 in hESC lines on feeder (a-d) or feeder-free (e-g) cultures. Single cell suspensions of hESC were fixed, permeabilized and stained with a monoclonal antibody against Oct-4. The labeled cells were detected with a FITC-conjugated anti-mouse antibody. The shaded histogram represents staining with the negative control and open histograms represent staining with Oct-4 mAb.

FIG. 4. Expression of cell surface markers on HES3 co-cultured with ΔE-MEF (top panel) or CM from ΔE-MEF (lower panel). Staining of cells with SSEA-4 (a, e), Tra-1-60 (b, f), Tra-1-81 (c, g) and alkaline phosphatase (d, h). For SSEA4 and Tra-1-60/81, colonies were subsequently stained with PE-conjugated secondary antibody. For AP, positive activity was characterized by a red precipitate that fluoresces using the rhodamine filter. Scale bar=100 μm.

FIG. 5. Section of a teratoma derived from HES-3 cells cultured for 14 passages (70 PD) under feeder-free conditions supplemented with CM from ΔE-MEF. Gut-like epithelium (a), neural epithelium (b), muscles (c), cartilage (d) and bone (e). Scale bar=20 μm.

FIG. 6. Cytogenetic analysis of undifferentiated HES-3 cells cultured on feeder (a) or feeder-free (b) conditions. Normal female karyotype (46 X,X) was observed for hESC from both cultures.

FIG. 7. RT-PCR analysis for the expression of HPV-E6, HPV-E7, Oct-4 and β-actin in ΔE-MEF (a) and HES-3 feeder-free cultures (b). Amplified products following RT-PCR were resolved on 1% agarose gels.

FIG. 8. Flow cytometry analysis of intracellular Oct-4 in HES-3 cells expanded on ΔE-MEF using triple flask (a) and cell factory (b). The shaded histogram represents staining with the negative control and open histograms represent staining with Oct-4 mAb.

### BEST METHOD OF CARRYING OUT THE INVENTION

### Materials & Methods

### Cell Culture

Human embryonic stem cell lines, HES-2 (46 X,X), HES-3 (46 X,X) and I-IES-4 (46 X,Y) were obtained from ES Cell International. The cells were cultured at 37° C./5% C02 either on mitomycin-C inactivated feeders (˜4×10⁴ cells/cm2) in gelatin-coated organ culture dish (co-cultures) or on matrigel-coated organ culture dishes supplemented with CM from feeders (feeder-free cultures). Media used for culturing hESC was KNOCKOUT (KO) medium which contained 85% KO-DMEM supplemented with 15% KO serum replacer, 1 mM L-glutamine, 1% non-essential amino acids and 0.1 mM 2-mercaptoethanol and 4-8 ng/ml of basic fibroblast growth factor (Invitrogen). Medium was changed daily and the cultures were passaged weekly following enzymatic treatment as previously described [4]. Culture dishes for feeder-free cultures were incubated at 40° C. overnight with matrigel (Becton Dickinson) diluted in cold KO-DMEM (1:30 dilution).

### Preparation of MEF and MEF Conditioned Media (CM)

Primary MEF were isolated from the fetuses of 129X1/SvJ mice (day 13.5 post coitum) using the methods described by Robertson et al [21]. Monolayers of primary MEF (passage 4) were cultured to confluency and treated with 10 μg/ml mitomycin-C for 2.5-3 h. Following treatment, cells were detached with 0.25% trypsin-EDTA and seeded onto organ culture dishes as described above in F-DMEM media. This media consists of 90% DMEM high glucose, 10% FBS, 2 mM L-glutamine, 25 U/ml penicillin and 25 μg/ml streptomycin (Invitrogen): Culture medium was changed to KO medium 24 h after seeding and allowed to equilibrate for an additional 24 h before adding hESC or collection of MEF-CM. For MEF-CM, culture dishes were seeded with 1.4×10⁵ cells/cm2 of mitomycin-C treated MEF and CM was collected every 24 h after KO medium was added into the dish. The CM was filtered (0.22 μm) and supplemented with an additional 8 ng/ml of recombinant human basic fibroblast growth factor (Invitrogen).

### Establishment of Immortalized Feeder Line, ΔE-MEF

Primary MEF (3×10⁵ cells) at passage 3 was seeded into 75 cm2 T-flask and allowed to adhere overnight in F-DMEM medium. The cells were then transduced with sterile filtered supernatant containing retroviruses from PA317 LXSN HPV16E6E7 or PA317 PXSV packaging cell lines (CRL-2203 and CRL-2202 respectively, ATCC) in the presence of 8 μg/ml of polybrene (Sigma-Aldrich) for 8 h at 37° C. [22]. Following that, the medium containing the virus was removed and replaced with fresh F-DMEM and incubated for 3 additional days. Transduced cells were then selected in the presence of 100 μg/ml of G418 (Sigma-Aldrich) for 14 days. The established feeder line, ΔE-MEF was routinely cultured in F-DMEM medium without antibiotics and stocks cryopreserved in 90% FBS and 10% DMSO.

### Growth Rates and Doubling Times

Single cell suspensions of MEF and hESC were harvested daily (days 1-6 after inoculation) following treatment with 0.25% trypsin-EDTA (Invitrogen). Trypan blue exclusion was used to determine the viable cell number and viability for each sample.

Graphs of viable cell number versus time were plotted in order to estimate the specific growth rate of cells during the exponential growth phase. From this, the doubling time (td) was calculated using the following equation, td=In(2)/μ, where μ is the specific growth rate (hr⁻¹).

### Flow Cytometry Analysis of Oct-4 Expression

Expression levels of the intracellular transcription, factor, Oct-4, in hESC populations were assessed by immunofluorescence using flow cytometry. Cells were harvested as a single cell suspensions using trypsin, fixed, permeabilized (Caltag Laboratories) and incubated with a mouse monoclonal antibody to Oct-4 (Santa Cruz) at a 1:20 dilution. Cells were then washed with 1% BSA/PBS, and incubated in the dark with a 1:500 dilution of goat α-mouse antibody FITC-conjugated (DAKO). After incubation, the cells were again washed and resuspended in 1% BSA/PBS for analysis on a FACScan (Becton Dickinson FACS Calibur). All incubations were performed at room temperature for 15 min. As a negative control, cells were stained with the appropriate isotype control.

### Immunocytochemistry

Cells were fixed in 4% paraformaldehyde at room temperature for 45 min and incubated with antibodies to SSEA-4 (neat, Developmental Studies Hybridomas Bank), Tra-1-60 and Tra-1-81 (30 μg/ml, Chemicon) at room temperature for 1 h. Localization of antibodies was visualized using goat α-mouse antibody FITC-conjugated (1:500 dilution; DAKO).

### Alkaline Phosphatase Staining

Alkaline phosphatase staining was performed using Vector Red Alkaline Phosphatase Substrate Kit 1 (Vector Laboratories) according to the manufacturer's protocol. Briefly, hESC were washed once in PBS followed by incubation in the dark with Vector Red substrate working solution for 45 min at room temperature. Rhodamine excitation and emission filters were used to visualize the reaction product.

### RNA Isolation and Reverse Transcription PCR (RT-PCR)

Total RNA was isolated from hESC and MEF using NucleoSpin RNA II. Kit from Macherey Nagel and quantified by ultraviolet spectrophotometry. Standard reverse transcription reactions were performed with 1 μg total RNA using oligo dT primers and ImProm II reverse transcriptase (Promega). The PCR was carried out with primers specific to Oct-4, β-actin, HPV 16 E6 and HPV 16 E7 (Table I). The cycling parameters used for amplification were 30 cycles of 95° C. for 1 min, 55° C. for 1 min and 72° C. for 1 min. This was followed by a final extension at 72° C. for 10 min. The amplified products were visualized on 1% agarose gels and stained with ethidium bromide.

### SCID Mouse Models

ΔMEF and hESC were enzymatically treated and ˜4-5×10⁶ cells were injected with a sterile 22 G needle into the rear leg muscle of 4 week old male SCID mice. Animals which developed tumours (˜9-10 weeks after injection) were sacrificed and the tumours were dissected and fixed in 10% formalin. Tumours were embedded in paraffin, sectioned and examined histologically after hematoxylin & eosin staining.

### Karyotyping

Karyotyping analysis was performed by the Cytogenetics Laboratories at the Dept of Obstetrics and Gynecology, NUS or KK Women's and Children's Hospital.

Scale-Up of hESC Co-Cultures

To scale-up the quantities of hESC, co-cultures were expanded on tissue culture flask (T75, 75 cm²), triple flask (TF, 500 cm²) and cell factories (CF, 632 cm²) (Nunc). The culture conditions used were similar to that described above. Mitomycin-C inactivated feeders were seeded on gelatinized culture surfaces at approximately 4×10⁴ cells/cm² for all surfaces. For passaging, cells were washed with phosphate-buffered saline+(PBS+) followed by incubation with 0.05% trypsin (Invitrogen) for 2-3 min at room temperature. The cells were dislodged from the flask by tapping and further dissociated to smaller clumps by gentle repeated pipetting. After which, trypsin was neutralized and the cell clumps were inoculated into new gelatinized flasks seeded with inactivated feeders at a ratio of 1:3.

### Results

Establishment and Characterization of Immortalized MEF Primary MEF at passage 3 were infected with retroviruses simultaneously encoding for HPV16-E6, HPV16-E7 and neomycin resistance (ΔE-MEF). As a negative control, cells were infected with a control retrovirus containing only neomycin resistance (AC-MEF).

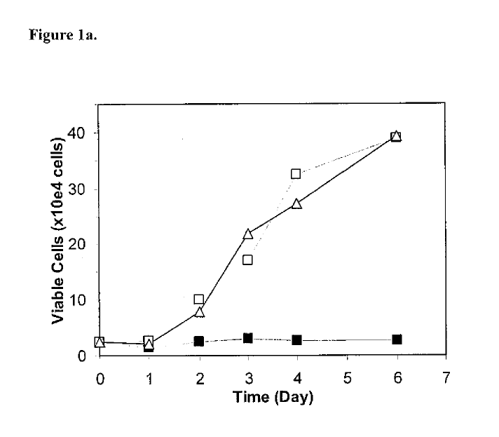

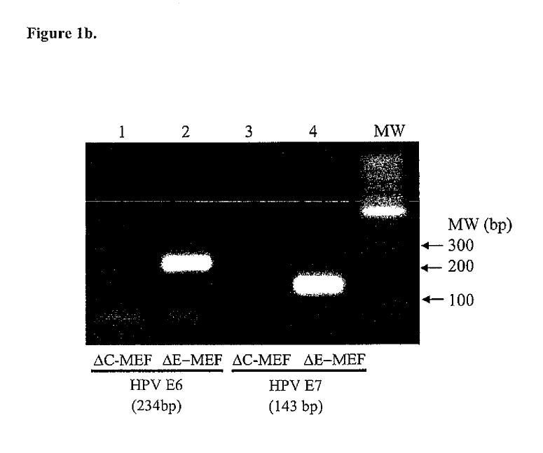

Four passages after infection (˜12 days post-infection, P7), ΔE-MEF continued to proliferate (FIG. 1a) with an approximate doubling time of 19.7 h. Furthermore, the growth rate and doubling time of ΔE-MEF was comparable to primary MEF at passage 4 (Table 2). In contrast, cells infected with the control virus (ΔC-MEF) at passage 7 showed signs of senescence because there was no significant increase in viable cell numbers over the identical 6-day period (FIG. 1a). After 45 passages (165 population doublings), ΔE-MEF continued to maintain similar growth kinetics compared to passage 7 (FIG. 1a) and is currently cultured beyond 70 passages without any decrease in proliferative capacity (data not shown). RNA from ΔE-MEF and AC-MEF at passage 7 was isolated and examined by RT-PCR for the expression of the E6 and E7 genes introduced as a result of the infection with the retrovirus. From FIG. 1b, it was apparent that ΔE-MEF expressed the mRNA for both E6 and E7 genes (lanes 2 and 4 respectively) whilst no expression was observed in AC-MEF (lanes 1 and 3), where the cells were infected with control viruses.

In order to determine whether ΔE-MEF was capable of forming tumors, following immortalization with E6 and E7, 5×10⁶ cells were injected into SCID mice. Animals were examined periodically and no palpable tumors were observed even after 16 weeks following injection of the cells (data not shown).

Growth and Morphology of hESC in Feeder and Feeder-free Conditions Using Immortalized MEF

Human ES cell line, HES-3 was routinely cultured either directly on primary MEF (co-cultures) or on matrigel supplemented with conditioned medium from primary MEF cultures (feeder-free). To evaluate whether the immortalized MEF line, ΔE-MEF, was able to support undifferentiated growth of hESC, HES-3 cells were seeded onto the respective conditions using ΔE-MEF instead.

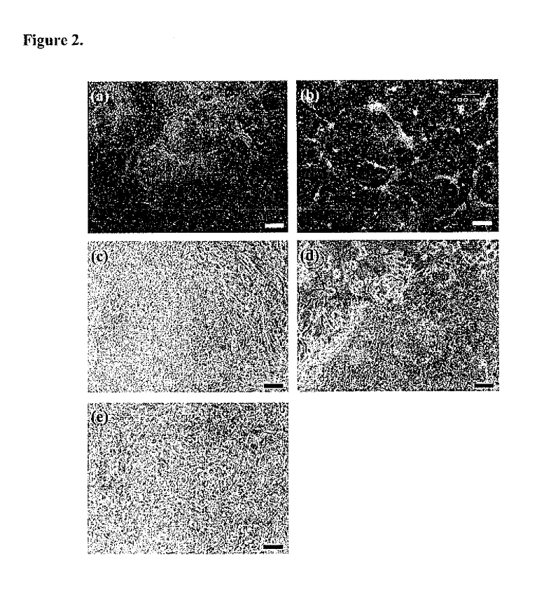

For co-cultures, clumps of HES-3 cells seeded onto ΔE-MEF expanded and formed colonies whilst pushing aside the feeders. This gave the colonies a very distinct border of fibroblastic cells surrounding the colonies (FIGS. 2a and 2c). Morphologically, hESC within the colonies remained tightly clustered and maintained a high nucleus to cytoplasmic ratio (FIG. 2e). For feeder-free cultures, despite the absence of feeders, hESC on matrigel, supplemented with ΔE-MEF conditioned medium continued to form distinct and compact colonies similar to hESC cultured on feeders (FIGS. 2b and 2d). Interestingly, differentiated fibroblast-like cells were observed surrounding the borders of the undifferentiated colonies however the density of these cells was significantly less compared to hESC (FIG. 2d). Under both feeder and feeder-free conditions, HES-3 cells continued to maintain undifferentiated hESC morphology for greater than 40 continuous passages (210 population doublings, PD). Similar morphologies were also observed for 2 additional hESC lines, HES-2 and HES-4 in feeder and feeder-free cultures for more than 10 passages (data not shown). In addition, the specific growth rate and doubling time of HES-3 cells in these two culture conditions were comparable (Table 2) and corresponded to published literature [4,23].

Characterization of Markers Unique to Undifferentiated hESC

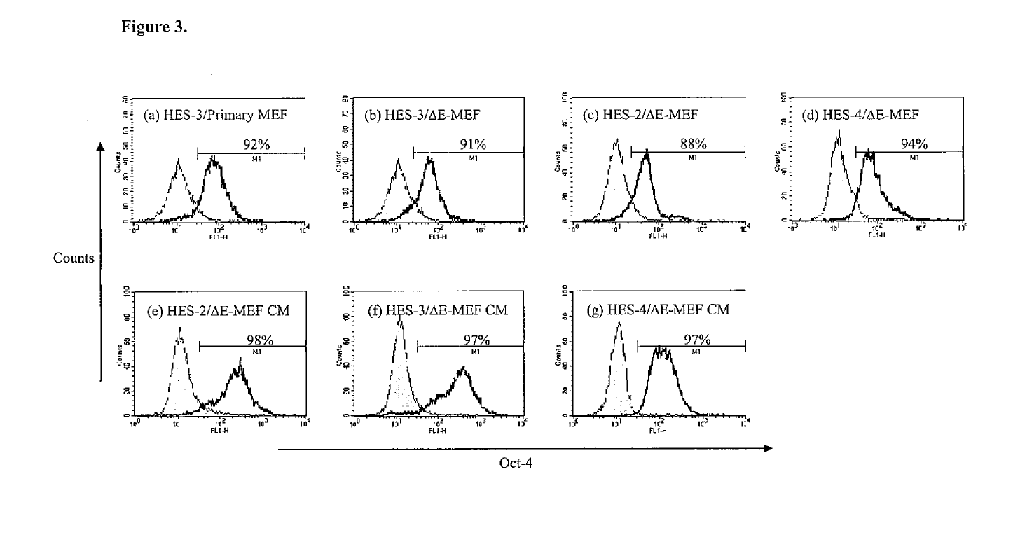

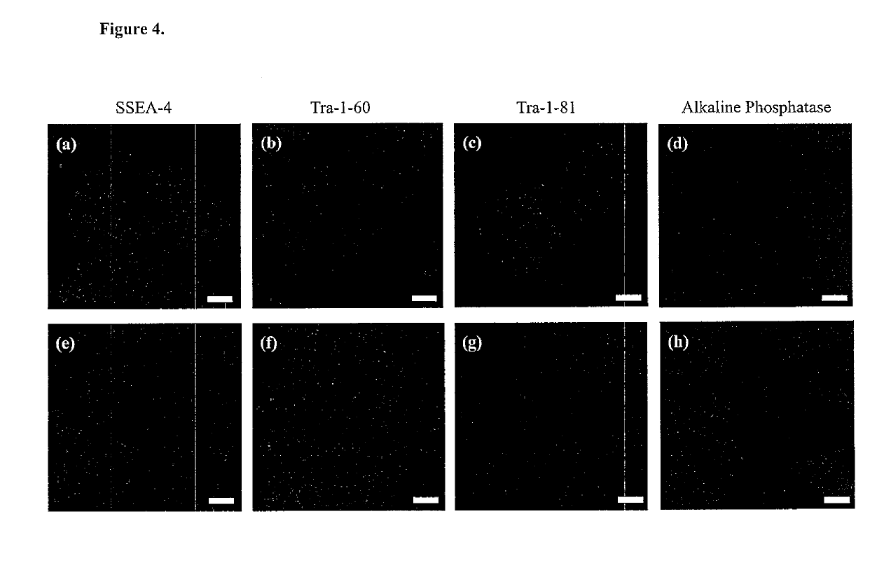

Undifferentiated hESC are characterized by the expression of a series of markers such as the intracellular transcription factor, Oct-4, and surface markers such as SSEA-4, Tra-1-60, Tra-1-81. Using flow cytometry, we compared the expression of Oct-4 in all 3 hESC lines in both feeder and feeder free conditions (FIG. 3). Like HES-3 cells cultured on primary MEF (FIG. 3a), >88% of HES-2, HES-3 and HES-4 cells (FIGS. 3c, 3b and 3d respectively) cultured on ΔE-MEF stained positive for Oct-4. Similarly, when maintained in feeder-free condition with CM from ΔE-MEF, >97% of cells (all 3 hESC lines) were positive for Oct-4 (FIGS. 3e-3g). Furthermore, HES-3 cells cultured on ΔE-MEF (FIG. 4, top panel) or ΔE-MEF CM (FIG. 4, lower panel) stained positive for SSEA4 (FIGS. 4a, 4e), Tra-1-60 (FIGS. 4b, 4f), Tra-1-81 (FIGS. 4c, 4g) and had high alkaline phosphatase activity (FIGS. 4d, 4h).

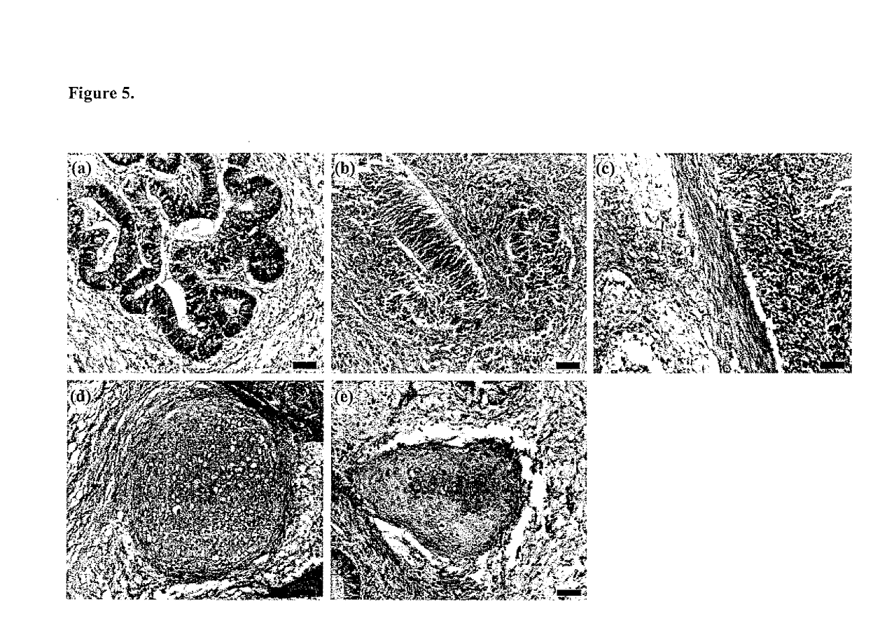

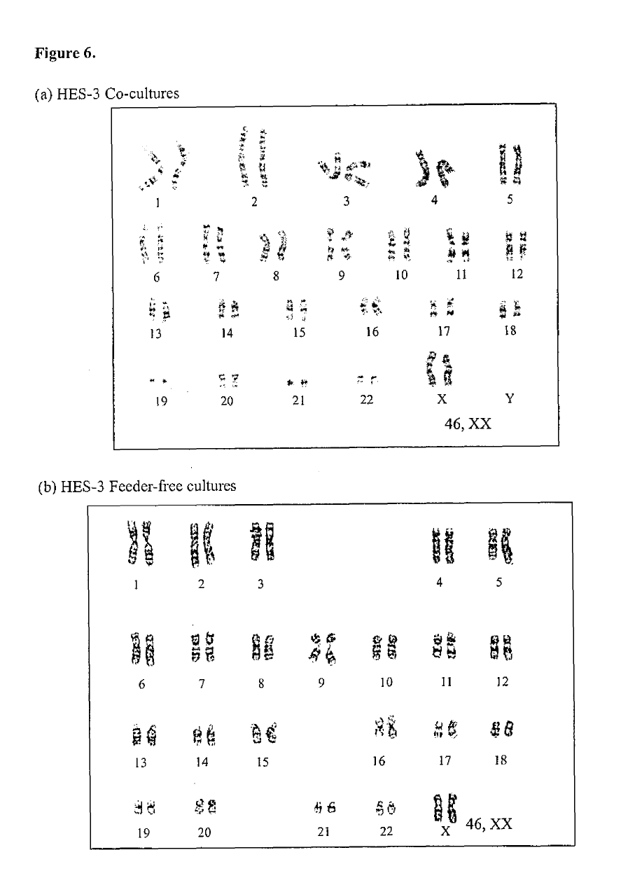

In order to evaluate the potential to form differentiated tissues in vivo, HES-3 cells maintained on feeder or feeder-free cultures using ΔE-MEF were injected into SCID mice. Ten weeks following injection, teratomas were observed in all conditions. The tumors were sectioned and examined histologically. From the sections, representative tissues to the 3 embryonic germ layers were identified, including gut-like epithelium (endoderm, FIG. 5a), neural epithelium (ectoderm FIG. 5b), muscles, cartilage and bone (mesoderm, FIGS. 5c-5e). Furthermore, cytogenetic examination was carried out on HES-3 cells from both feeder and feeder-free cell populations after at least 25 continuous passages (130 PD) (FIGS. 6a and 6b respectively). Results from the analysis showed that the HES-3 cells continue to maintain normal karyotype (46 X,X) under both culture conditions.

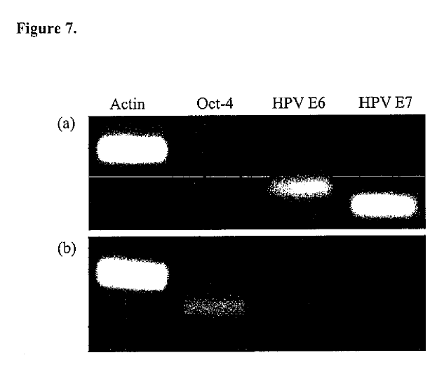

Lastly, since retroviruses were used to immortalize the primary MEF, it was important to establish that viruses were not produced by ΔE-MEF, which could then infect the hESC with the E6 and E7 antigens. RT-PCR did not detect the expression of both E6 and E7 antigens in HES-3 feeder-free cultures after 40 passages (FIG. 7b). Instead, the expression of Oct-4 was detected confirming that the hESC remained undifferentiated. As a control, RT-PCR was also performed on ΔE-MEF (FIG. 7a). Here, expression of both E6 and F7 antigens by the feeders were detected but not Oct-4.

### Scale-Up of HES-3 Co-Cultures Using ΔE-MEF

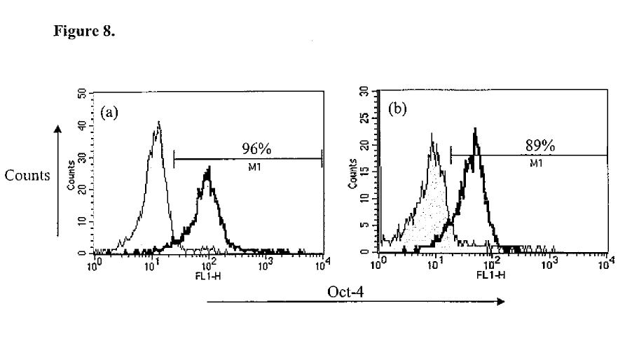

To demonstrate that ΔE-MEF could be used for the scale-up of undifferentiated hESC, co-cultures were expanded from organ culture dishes to tissue culture flasks. Cells from tissue culture flasks were then used as inoculum for hESC expansion into triple flasks and cell factories. From Table 3, the total cell yield obtained after 7 days in these large culture vessels were approximately 2−3×10⁸ cells. It is also apparent that despite the increase in the surface area by 208× and 263× for triple flask and cell factory respectively compared to an organ culture dish, the cell density was maintained at ˜0.37-0.47×10⁶ cells/cm². Moreover, 89%-96% of expanded hESC population in triple flask and cell factory continue to stain positive for Oct-4 (FIGS. 8a and 8b respectively). Taken together, these results suggest that it is possible to expand undifferentiated hESC on ΔE-MEF co-cultures to significant quantities.

TABLE 1
Primer Sequences for RT-PCR

| | | | |
|---|---|---|---|
| | | Product | |
| | | Size | |
| Primer | Sequence (5′-3′) | (bp) | Ref. |

| | | | |
|---|---|---|---|
| HPV16-E6-F | GCAACAGTTACTGCGACGTG | 234 | [11] |
| HPV16-E6-R | GGACACAGTGGCTTTTGACA | 234 | [11] |
| HPV16-E7-F | GATGGTCCAGCTGGACAAGC | 143 | [11] |
| HPV16-E7-R | GTGCCCATTAACAGGTGTTC | 143 | [11] |
| Oct-4-F | CGRGAAGCTGGAGAAGGAGAAGCTG | 241 | [2] |
| Oct-4-R | AAGGGCCGCAGCTTACACATGTTC | 241 | [2] |
| β-Actin-F | TGGCACCACACCTTCTACAATGAGC | 396 | [2] |
| β-Actin-R | GCACAGCTTCTCCTTAATGTCACGC | 396 | [2] |

TABLE 2

Specific growth rates and doubling
times of feeders, hESC co-cultures
and feeder-free cultures

| | | Specific Growth | Doubling |
|---|---|---|---|
| hESC Line | Feeder | Rate/μ (h⁻¹) | Time/t_{d}(h) |
| — | MEF (P4) | 0.049 | 20.4 |
| — | ΔE-MEF (P7) | 0.051 | 19.7 |
| HES-3 | ΔE-MEF | 0.022 | 31.8 |
| HES-3 | — | 0.021 | 32.7 |
| | (Feeder-free | | |
| | ΔE-MEF CM) | | |

TABLE 3

Total cell yield and cell density of hESC
co-cultures in different culture platforms

| | | | |
|---|---|---|---|
| | Surface | Total | Cell Density |
| Culture | Area | Cell Yield | (× 10⁶ |
| Platform | (cm²⁾ | (× 10⁶ cells) | cells/cm²) |

| | | | |
|---|---|---|---|
| Organ Culture | 2.4 | 1.0 | 0.42 |
| Dish | | | |
| Tissue Culture | 75.0 | 29.4 | 0.39 |
| Flask | | | |
| Triple Flask | 500.0 | 187.5 | 0.37 |
| Cell Factory | 632.0 | 295.3 | 0.47 |

### DISCUSSION

Feeder cells are an essential component in the culture of hESC. Unlike mouse embryonic stem cells, hESC are grown either as co-cultures in direct contact with feeders or in feeder-free cultures supplemented with feeder-CM. Under these conditions, hESC continue to maintain an undifferentiated phenotype. However, a major limitation is that the feeders currently used in hESC work are derived from primary tissues of either mouse or human sources. As normal somatic cells, they have a limited lifespan in culture and will senesce after a finite number of replications. The transduction of primary cells with HPV16 E6 and E7 antigens has been shown to extend the lifespan of human fibroblast cells beyond 200 population doublings [19].

In this study, we demonstrated that the lifespan of primary MEF can be extended from 7-9 passages to beyond 70 passages (250 population doublings) by infection with HPV16 E6 and E7. The expression of both E6 and E7 antigens were confirmed by RT-PCR. It has been previously reported that cells immortalized using genes such as SV40, E6/E7 enter into a crisis phase where the majority of cells senesce. From this population, cells that continue to divide go on to form the immortalized cell line [24]. Interestingly, this was not observed with ΔE-MEF. The bulk of the cells continued to proliferate beyond their normal lifespan and was resistant to antibiotic selection with G418 following infection with the retroviruses. In vivo, intramuscular injection of ΔE-MEF into SCID also did not result in any palpable tumors even 16 weeks after injection. This confirms that ΔE-MEF has not become tumorigenic after immortalization. In contrast, an equivalent amount of hESC injected formed teratomas of approximately 1-2 cm in diameter 9-10 weeks post-injection.

The major concern for the use of E6 and E7 antigens to immortalize cells is that although immortalization can be achieved by the degradation of p53 and retinoblastoma proteins, it has been reported that E6 and E7 may also affect other pathways and cellular functions in the immortalized cell. Murvai et al [25] observed that HPV16 E7 also repressed the activity of the transforming growth factor-β2 (TGF-β2) promoter in NIH/3T3 cells. In human keratinocytes, the over-expression of E6 and E7 resulted in a decrease in the expression of TGF-β2 mRNA and the secretion of biologically active TGF-β2 [26]. On the other hand, HPV 16 E6 significantly induced the activity of TGF-β1 promoter by six-fold however this effect was cell-type specific and was only observed in the fibroblast cells' but not in epithelial cells [27].

In the context of this study, E6 and E7 immortalization has not altered the ability of ΔE-MEF to support undifferentiated hESC growth. We observed that 3 hESC lines readily adapted to these feeders and maintained the typical morphology of undifferentiated hESC cultures both in feeder and feeder-free cultures. The hESC also continued to express pluripotent markers, including Oct-4, SSEA4, Tra-1-60, Tra-1-81 and alkaline phosphatase. After 25 passages, the cells retained a stable karyotype and were able to differentiate to form teratomas in SCID mice. In addition, RT-PCR analysis of mRNA from HES-3 feeder-free cultures confirmed that the cells remained positive for Oct-4 but negative for E6 and E7 antigens. The latter result is significant because it proves that ΔE-MEF is safe for use with hESC and there is not a risk of transfer of these antigens from the feeders to the hESC. In our efforts to produce significant quantities of undifferentiated hESC, we demonstrated that it was feasible to scale-up co-cultures of HES-3 cells using ΔE-MEF in large culture vessels, such as cell factory (˜2-3×10⁸ cells). Despite the increase in surface area by 208-263× compared to an organ culture dish, it was possible to obtain comparable densities of hESC after 7 days of culture without compromising on the quality of the cells.

More recently, our group has also successfully immortalized 3 human foreskin fibroblast (hF) lines previously shown to support hESC expansion using the methods described here [4]. Like ΔE-MEF, the immortalized hF line, ΔE-Hs68, retained its ability to support the proliferation of undifferentiated HES-3 cells in feeder co-cultures (>26 passages) and feeder-free cultures (>16 passages). The doubling time of the HES-3 cells were 34.5 h and 36.4 h respectively compared to 33.3 h for HES-3 cells on primary Hs68 co-cultures. The hESC also continued to stain positive for Oct-4, GCTM-2, SOX-2, SSEA-4, Tra-1-60 and Tra-1-81 (results not shown).

In conclusion, these results suggest that Immortalizing cells using E6 and E7 is a generic strategy that can be applied to both mouse and human feeders. The ability of feeders to proliferate beyond their normal lifespan and yet retain the ability to support hESC expansion is beneficial because it will eliminate the need for frequent preparations of primary feeders. This consistent source of feeders will facilitate reproducibility in results especially in the scale-up of hESC in both feeder and feeder-free conditions and it will also reduce the problems associated with batch to batch variation in feeder quality. Moreover, these immortal feeder lines cells can be screened to ensure that they are free of potential pathogens which can be transferred from the feeders to the hESC.

### REFERENCES

1. Thomson J A, Itskovitz-Eldor J, Shapiro SS et al. Embryonic stem cell lines derived from human blastocysts. Science 1998; 282: 1145-1147.
2. Richards M, Fong C Y, Chan WK et al. Human feeders support prolonged undifferentiated growth of human inner cell masses and embryonic stem cells. Nat Biotechnol 2002; 20: 933-936.
3. Reubinoff B E, Pera M F, Fong C Y et al. Embryonic stem cell lines from human blastocysts: somatic differentiation in vitro. Nat Biotechnol 2000; 18:399-404.
4. Choo A B, Padmanabhan J, Chin AC et al. Expansion of pluripotent human embryonic stem cells on human feeders. Biotechnol Bioeng 2004; 88:321-331.
5. Amit M, Margulets V, Segev H et al. Human feeder layers for human embryonic stem cells. Biol Reprod 2003; 68:2150-2156.
6. Richards M, Tan S, Fong C Y et al. Comparative evaluation of various human feeders for prolonged undifferentiated growth of human embryonic stem cells. Stem Cells 2003; 21:546-556.
7. Hovatta O, Mikkola M, Gertow K et al. A culture system using human foreskin fibroblasts as feeder cells allows production of human embryonic stem cells. Hum Reprod 2003; 18:1404-1409.
8. Carpenter M K, Rosier E S, Fisk G J et al. Properties of four human embryonic steni cell lines maintained in a feeder-free culture system. Dev Dyn 2004; 229:243-258.
9. Xu C, Inokuma M S, Denham J et al. Feeder-free growth of undifferentiated human embryonic stem cells. Nat Biotechnol 2001; 19:971-974.
10. Bryan T M, Reddel R R. SV40-induced immortalization of human cells. Crit Rev Oncog 1994; 5:331-357.
11. Gudjonsson T, Villadsen R, Nielsen H L et al. Isolation, immortalization and characterization of a human breast epithelial cell line with stem cell properties. Genes Dev 2002; 16:693-706.
12. Jha K K, Banga S, Palejwala V et al. SV40-Mediated immortalization. Exp Cell Res 1998; 245: 1-7.
13. Sugimoto M, Ide T, Goto M et al. Reconsideration of senescence, immortalization and telomere maintenance of Epstein-Barr virus-transformed human B-lymphoblastoid cell lines. Mech Ageing Dev 1999; 107:51-60.
14. Yamamoto A, Kumakura S, Uchida M et al. Immortalization of normal human embryonic fibroblasts by introduction of either the human papillomavirus type 16 E6 or E7 gene alone. Int J Cancer 2003; 106:301-309.
15. Bodnar A G, Ouellette M, Frolkis M et al. Extension of life-span by introduction of telomerase into normal human cells. Science 1998; 279:349-352.
16. Xu C, Jiang J, Sottile V et al. Immortalized fibroblast-like cells derived from human embryonic stem cells support undifferentiated cell growth. Stem Cells 2004; 22:972-980.
17. Klingelhutz Al, Foster S A, McDougall J K. Telomerase activation by the E6 gene product of human papillornavirus type 16. Nature 1996; 380:79-82.
18. Boyer S N, Wazer D E, Band V. E7 protein of human papilloma virus-16 induces degradation of retinoblastoma protein through the ubiquitin-proteasome pathway. Cancer Res 1996; 56:4620-4624.
19. Shiga T, Shirasawa H, Shimizu K et al. Normal human fibroblasts immortalized by introduction of human papillomavirus type 16 (HPV-16) E6-E7 genes. Microbiol Immunol 1997; 41:313-319.
20. Halbert C L, Demers G W, Galloway D A. The E7 gene of human papillomavirus type 16 is sufficient for immortalization of human epithelial cells. J Virol 1991; 65:473-478.
21. Robertson, E. J. Embryo-derived stem cell lines. In: Robertson El, ed. Teratocarcinornas and embryonic stem cells. Oxford: IRL Press, 1987: 71-112.
22. Wazer D E, Liu X L, Chu Q et al. Immortalization of distinct human mammary epithelial cell types by human papilloma virus 16 E6 or E7. Proc Natl Acad Sci USA 1995; 92:3687-3691.
23. Cowan C A, Klimanskaya I, McMahon I et al. Derivation of embryonic stem-cell lines from human blastocysts. N Engl J Med 2004; 350: 1353-1356.
24. Yeager T R, Reddel R R. Constructing Immortalized human cell lines. Curr Opin Biotechnol 1999; 10:465-469.
25. Murvai M, Borbely A A, Konya I et al. Effect of human papillomavirus type 16 E6 and E7 oncogenes on the activity of the transforming growth factor-beta2 (TGF-beta2) promoter. Arch Virol 2004; 149:2379-2392.
26. Nees M, Geoghegan J M, Munson P et al. Human papillomavirus type 16 E6 and E7 proteins inhibit differentiation-dependent expression of transforming growth factor-beta2 in cervical keratinocytes. Cancer Res 2000; 60:4289-4298.
27. Dey A, Atcha I A, Bagchi S. HPV16 E6 oncoprotein stimulates the transforming growth factor-beta 1 promoter in fibroblasts through a specific GC-rich sequence. Virology 1997; 228: 190-199.

1-36. (canceled)

37. An immortalized feeder cell line.

38. The immortalized feeder cell line of claim 37 which is derived from an embryonic fibroblast.

39. The immortalized feeder cell line of claim 37 which is derived from a mouse embryonic fibroblast.

40. The immortalized feeder cell line of claim 37 which is derived from a mouse embryonic fibroblast and exhibits one or more of, degradation of the p53 tumor suppressor protein, up-regulation of c-myc expression, activation of telomerase and degradation of pRb.

41. The immortalized feeder cell line of claim 37 which is derived from an embryonic fibroblast by introduction of the E6 and E7 genes from HPV16.

42. The immortalized feeder cell line of claim 41, wherein the E6 and E7 genes are introduced into embryonic fibroblast cells by transduction.

43. The immortalized feeder cell line of claim 42, wherein the embryonic fibroblast cells are infected with retrovirus vectors encoding the E6 and E7 genes from HPV16.

44. The immortalized feeder cell line of claim 43, wherein the cells continue to proliferate beyond their normal lifespan and are resistant to antibiotic selection with G418 following infection with the retroviruses.

45. The immortalized feeder cell line of claim 43, wherein the cells do not become tumorigenic after immortalization.

46. The immortalized feeder cell line of claim 45, wherein in vivo, intramuscular injection of the cells into a SCID mouse does not result in any palpable tumors at least 16 weeks after injection.

47. The immortalized feeder cell line of claim 37, wherein the cell line proliferates beyond 7, 8 or 9 passages.

48. The immortalized feeder cell line of claim 47, wherein the cell line proliferates in vitro beyond 70 passages and does not gain any tumorigenic phenotype.

49. The immortalized feeder cell line of claim 37, wherein the cell line supports hESC, and wherein the hESC cells continue to maintain characteristic undifferentiated morphology for >40 passages both in co-culture and in feeder-free cultures supplemented with CM, and continue to express the pluripotent markers, Oct-4, SSEA-4, Tra-1-60, Tra-1-81, alkaline phosphatase, and maintain a normal karyotype, and form teratomas with tissues representative of the 3 embryonic germ layers when injected into SCID mice.

50. The immortalized feeder cell line of claim 37, wherein the cell line supports undifferentiated hESC growth.

51. The immortalized feeder cell line of claim 37, wherein hESC readily adapt to the immortalized feeder cell line and maintain a typical morphology of undifferentiated hESC cultures both in feeder and feeder-free cultures.

52. The immortalized feeder cell line of claim 51, wherein the hESC continue to express pluripotent markers, including Oct-4, SSEA-4, Tra-1-60, Tra-1-81 and alkaline phosphatase.

53. The immortalized feeder cell line of claim 51, wherein after 25 passages, the hESC retain a stable karyotype and are able to differentiate to form teratomas in SCID mice.

54. The immortalized feeder cell line of claim 51, wherein RT-PCR analysis of mRNA from hESC feeder-free cultures confirms that the cells remain positive for Oct-4 but negative for E6 and E7 antigens.

55. Primary mouse embryo fibroblasts which are immortalized by the over-expression of E6 and E7 antigens.

56. A cell line ΔE-MEF as herein described.

57. The cell line of claim 56 which is cultured in a suitable culture medium.

58. A composition comprising the immortalized feeder cell line of claim 37 in a suitable carrier or diluent.

59. A conditioned medium produced from growth of the immortalized feeder cell line of claim 37.

60. The conditioned medium of claim 59, which is used in conjunction with extracellular matrices, including matrigel.

61. A method of culturing a stem cell, comprising the step of co-culturing the stem cells with feeder cells, wherein the feeder cells are the immortalized feeder cell line of claim 37.

62. The method of claim 61, wherein the stem cell is a human stem cell.

63. The method of claim 62, wherein the stem cell is a human embryonic stem cell.

64. A method of culturing a stem cell, comprising the step of culturing the stem cell in the conditioned medium of claim 59.

65. The method of claim 64, wherein the stem cell is a human stem cell.

66. The method of claim 65, wherein the stem cell is a human embryonic stem cell.

67. The method of claim 61 which produces scaled-up quantities of undifferentiated hESC in culture vessels, including cell factories.

68. The method of claim 67, wherein the quantity of undifferentiated hESC is >10⁸ cells.

69. A stem cell cultured by the method of claim 61.

70. The stem cell of claim 69 which is a human stem cell.

71. The stem cell of claim 69 which is a human embryonic stem cell.
